(19) United States
(12) Patent Application Publication
(10) Pub. No.: US 2013/0129659 A1
(43) Pub. Date: May 23, 2013

(54) COMPOSITE ENTEROSORBENT

(71) Applicants: Vadim Kozlovskyi, Kyiv (UA); Yuriy Tolcheyev, Zugres (UA)

(72) Inventors: Vadim Kozlovskyi, Kyiv (UA); Yuriy Tolcheyev, Zugres (UA)

(21) Appl. No.: 13/611,661

(22) Filed: Sep. 12, 2012

(30) Foreign Application Priority Data
Nov. 23, 2011 (UA) 2011 13802

(51) Int. Cl. A61K 31/80; A61P 31/00; A61P 39/02; A61P 1/00

(52) U.S. Cl. 424/78.01

(57) ABSTRACT

A composite enterosorbent has a substrate in the form of a sorbent based on hydrogel of polymethylsiloxane. For the purpose of complex normalization of the intestinal microflora it further comprises a polysaccharide selected from the group including lactulose, inulin, fructooligosaccharides, alginic acid in the form of its pharmaceutically acceptable salts, acacia, pectin, chitosan, lignin. Respectively, the preparation comprises 0.1 to 10 parts by weight of polysaccharides per 1 part by weight of hydrogel of polymethylsiloxane.

### FIELD OF THE INVENTION

The present invention relates to compositions of preparations based on derivatives of methyl-silicic acid and polysaccharides. These preparations are indicated for the use in treatment of diseases associated with the intoxication syndrome, particularly gastrointestinal diseases, especially those caused by pathogenic microorganisms and/or toxins and accompanied with alterations in gastrointestinal flora.

### PRIOR ART

According to modern teachings intestinal dysbiosis (ID) implies quantitative and qualitative alterations in the normal flora of an organ with deterioration of its biological functions and consistent overgrowth of opportunistic enterobacteria, which results from a number of unfavorable factors.

The English language publications refer to the term <<bacterial overgrowth syndrome>>, while in German literature the term <<bakterielle fehlbesiedlung>>, meaning erroneous, abnormal colonization of bacteria, is used. Obviously, in such a case it is the specific connotation that matters rather then the term chosen. Thus, this implies changes in flora of the lumen of the small intestine due to abnormal colonization of bacteria, as well as due to drastic decrease in the number of normal microflora of the genus Bifidobacterium, Escherichia Coli and simultaneous increase in the contents of colibacillus with weak enzymatic properties, lactose-negative enterobacteria, Candida genus fungi, etc. in the colon.

Obligate bacteria (bifidobacteria, bacteroids) permanently reside in the normal microflora and are responsible for metabolic processes and protection of the host from infectious agents. They constitute about 95-97% of the intestinal microflora. Facultative microorganisms (lactobacteria, colibacillus, enterococcus) with specific gravity of 4-5% are opportunistic microorganisms since they are often detected in healthy humans. However, in case of low immunity such microorganisms develop aggressive properties and cause the development of certain diseases. The remaining aerobic saprophytic opportunistic flora (Klebsiella, Proteus, yeasts, Clostridia, staphylococcus, etc.) constitutes less than 1% from the total number of microorganisms.

Thus, the majority of microorganisms in the colon are attached to the intestinal wall where they form microcolonies protected from exposure by a biofilm consisting of exopolysaccharides of microbial origin and mucine, which is a secreta of goblet cells (exopolysaccharide mucine matrix which serves as a specific placenta for microbal associations). The number of bacteria in the lumen of the colon (in the state of “free floating”) is much fewer than that of the localized microorganisms attached to the walls.

The frequency of ID in the population of Ukraine is 20 to 40% on the average, and reaches even up to 50% in children.

Among a great number of causes resulting in the development of ID in children and teens, those, which result in this condition in 100% of cases, include infections of gastrointestinal tract, particularly secretory and invasive (inflammatory) diarrhea, loose, as well as non-infectious diseases of the digestive system (chronic gastroduodenitis, pathologies of hepatobiliary system, etc.). As figuratively stated by professor S. A. Kramarev, << . . . each child that has suffered from an acute intestinal infection recovers from this “in the state of intestinal dysbiosis”.

The ID related to acute and chronic intestinal infections is based on a number of pathogenic mechanisms, the most important of which include impaired mucous membrane integrity, changes in intestinal motility and the development of the malabsorption syndrome, namely impaired nutritive absorption. This syndrome is to a large extent due to the imbalance in the normal intestinal flora since the latter is directly involved in the so-called parietal digestion. The process is based on that the surface of the small intestine mucous membrane villi has a large number of microvilli on which enzymes and human-specific microorganisms which assist in digestive reactions are adsorbed. At the same time, it has been shown that parietal digestion takes about 80% of the time necessary for the efficient digestive process. It is evident that any disruption in the state of normal microflora may result in digestive disorders with all possible outcomes, such as signs of chronic intoxication by suboxidized metabolites, anemia, hypovitaminosis, dehydration, cachexia, etc.

Disruption in the habitat for microorganisms in the intestine may result in eubiotic imbalance which promotes colonization of intestine with exogenous (alien/extrinsic for the body) microorganisms. As a result, the opportunistic microflora develops aggressive properties. Under these new and unusual conditions mass mortality of microorganisms occurs, which is accompanied by the release of endotoxins which, when absorbed into the blood, may result in toxicosis or, in especially severe cases, in shock.

Such changes distort metabolism in the mucous membrane cells of the small intestine, increase the activity of lipid peroxidation, which destabilizes cells structure. Activation of enzymatic proteolysis systems destructively affects intestinal cells membranes. A mucous membrane without the protective action of microvilli of the brush border becomes susceptible to microbal invasion, while the loss of parietal digestion aggravates dysfunction in secretory and resorbtive activities of the small intestine. As a result, the intestinal cavity accumulates large amounts of liquids and gases, which, in its turn, causes superdistension of the bowel, blocks normal blood inflow and outflow, thus promoting hypoxia, and is accompanied with dysfunction of both the intestines (with the development of the specific symptoms), and the body as a whole. Therefore, treatment of such conditions is a crucial task in the medicine.

A separate factor which plays an important role in the development of dysbiosis and toxicosis is the use of antibiotics. The statistics shows that 100% of people in the developed countries have taken antibiotics. However, the frequency of the development of dysbiosis following the treatment with antibiotics has not been confirmed. It is believed that it reaches the rate of 80%. According to professor S. A. Kramarev, in the background of treatment with antibiotics children exhibit a number of side effects on the part of gastrointestinal tract, particularly one third of the patients who have taken antibiotics for an extended period of time develop the diarrhea syndrome in one form or another, 66% experience stomach pain, 27%—bloating, 16%—emesis.

In the conditions of surgical in-patient units or in other cases when broad-spectrum antibiotics are used in the course of treatment, patients develop the so-called antibiotic-associated diarrhea (AAD) or antibiotic-associated colitis caused by Clostridium difficile. The frequency of occurrence of AAD depends on the type of the administered antibiotic and, according to different estimates, is in the range from 2% to 30%. Taking into account the number of patients taking antibiotics, this, obviously, is a serious scientific problem.

It has been proven that almost all antibiotics can cause diarrhea. However, there are the most “vicious” causative agents which result in this syndrome. As is known from medical literature, diarrhea is most frequently developed in the background of administration of clindamicin and the combination of amoxicillin with clavulanic acid.

It should be borne in mind that in the course of treatment with antibiotics, dysbiotic changes are the most apparent, and the state of eubiosis in the intestines does not restore for an extended period of time.

Therefore, it is obvious that correction of the state in the intestines is, first of all, necessary in case of various contagious diseases which are accompanied with intoxication, and, in the second place, upon treatment with antibiotics, which is particularly the case with children and medically fragile people.

The main targets in the correction of ID, including that following an acute intestinal infection, include fast elimination of toxins and restoration of population levels of the main representatives of normal anaerobic microflora of the intestine and its motility, as well as increasing immunobiological resistance of the body. In view of this, it is believed that the correction of dysbiotic disorders in the intestine will not only result in eubiosis, but will also remove the regular signs of the accompanying secondary immunodeficiency.

Traditionally, to this end use is made of avirulent strains of microorganisms which form the basis of the normal intestinal flora, for example respective strains of lactobacteria or bididobacteria, or their consortia, colibacillus and certain yeasts.

Thus, for example, Eurasian patent no 002614 of Jun. 27, 2002 discloses data on a consortium of antagonist strains of bifidobacteria and a new strain B. bifidum No. 791/BAG, which are less sensitive to antibiotics, but efficiently restore intestinal microflora. UA patent no. 24987 A (published on Dec. 25, 1998, Bulletin no. 6) discloses preparation of a food additive which restores intestinal microflora by incubation of lacto- and bifidobacteria together with polysaccharide vehicles. A similar process for the obtaining of cultures efficient in restoration of normal microflora is disclosed in the RU patent no. 2048123, (published on Nov. 20, 1995).

However, neither lacto-, nor bifidobacteria as such are capable of complete restoration of the state of eubiosis in the intestine, as well as of exhibiting antagonistic effect on other pathogenic microorganisms, for example S. aureus. From this point of view, supplementing a diet or a course of treatment or prevention of the development of intestinal dysbiosis with preparations based on a single culture or even a consortium of several cultures like, for example, the preparation marketed in Ukraine under the trade mark “BIFI-form”, limits the antimicrobial effect since these cultures are not capable of superseding all pathogenic or opportunistic microorganisms and totally restoring the microflora in a human's body.

One can not overlook the fact that efficiency of a number of biopharmaceuticals used for the correction of dysbiosis (probiotics), which are based on cultures representing normal intestinal microflora, is notably decreased due to a number of objective circumstances. Particularly, bacteria weakened as a result of drying, are susceptible to the active influence of pH medium in gastric acid and its digestive enzymes, which requires that such preparations are taken for an extended period of time in order to completely restore their biological activity.

Other strains of microorganisms which are traditionally used for the treatment of dysbiotic disorders also demonstrate similar effects.

For example, the yeasts Saccharomyces boulardii are capable of suppressing growth of pathogenic and opportunistic microorganisms and fungi which damage intestinal biocenosis, such as: Clostridium difficile, Clostridium pneumoniae, Staphilococcus aureus, Pseudomonas aeruginosa, Candida krusei, Candida pseudotropical, Candida albicans, Salmonella typhi, Salmonella enteritidis, Ecsherichia coli, Shigella dysenteriae, Shigella flexneri, Klebsiella, Proteus, Vibrio cholerae, as well as Enthamoeba hystolitica, Lambliae, Enterovirus, Rotavirus, etc.

UA patent no. 54103 A (published on Feb. 17, 2003, Bulletin no. 2/2003) discloses a method of restoration of normal microflora with the use of preparations comprising Saccharomyces boulardii in patients suffering from pancreatic disorders, while UA patent no. 62152 A (published on Dec. 15, 2003, Bulletin No. 12/2003) discloses a method of restoration of microflora in post-operation patients.

Saccharomyces boulardii have a number of substantial advantages over lacto- and bifidobacteria. Genetically determined resistance of Saccharomyces boulardii to the action of antibiotics explains a possibility of their simultaneous use with antibiotics to protect normal microbiocenosis of the digestive tract in the course of treatment with antibiotics. Furthermore, they are capable of disintegrating certain toxic substances which can accumulate in the intestine with damaged normal micorflora.

However, the use of the yeasts Saccharomyces boulardii themselves in the form of a powder placed within a capsule or tablet can not totally resolve the problem of dysbiosis which develops as a result of intestinal infections, surgery on the intestine, sepsis, different disorders accompanied with the intoxication syndrome, for example pancreatitis, irritated bowl syndrome, nonspecific ulcerative collitis, Crohn's disease, as a result of radiotherapy and chemotherapeutic agents used for the treatment of cancer, immunodeficiency states, for example the acquired immunodeficiency syndrome (AIDS), etc. This is explained by that therapeutic properties of Saccharomyces boulardii as such also deteriorate under the influence of aggressive factors, such as gastric acid containing hydrochloric acid, and bile acids which can damage Saccharomyces boulardii membranes.

Therefore, attempts have been made to use combinations of probiotics. The known preparation “Linex” comprises bifidobacteria, lactobacillus and enterococcus, as well as lactose. This facilitates normalization of microflora since, according to scientific data, restoration of a biofilm on a mucous membrane requires no less than 100,000 of beneficent bacteria per one square centimeter of the mucous membrane in the gastrointestinal tract. By coating mucous membranes, such a biofilm provides sustained protection from various infections and viruses. This biofilm may even be called a basis for the immune, hemogenic, vitamin-forming, digestive, enzymatic, hormonal functions of the gastrointestinal tract.

However, neither the individual use of eubiotics, nor their use in consortia does not provide the desired result since, as is known, their use in dry state in the form of tablets or capsules results in colonization of only 100 bacterial units per square centimeter of the mucous membrane in the gastrointestinal tract, which is much less than needed (op cit.: [symbol] M. A. [symbol] B [symbol] [symbol] [symbol] /M. A. [symbol] , [symbol] . [symbol] // [symbol] [symbol] ( [symbol] Consilium Medicum).—2008.—No. 1.—C. 50-52./M. A. Livzan Probiotics in the practice of medical practitioners/M. A. Livzan, M. B. Kostenko//Gastroenterology (annex Consilium Medicum). 2008. Issue no. 1, pages 50-52) Furthermore, the use of probiotics does not solve the problem of fast deintoxication which is vital in case of dysbiosis.

Recent studies have shown that for the purpose of normalization of microflora equally useful may be not only microorganisms (probiotics), but also substances which promote growth in the existing normal microflora, namely substances serving as substrates for normal microorganisms in the body. Such substances are called prebiotics. Prebiotics are defined as non-digestible substances which are beneficial to health due to arbitrary stimulation of the growth and/or metabolic activity of one or several groups of bacteria present in the colon, resulting in normalization of their ratio (op cit.: [symbol] [symbol] . [symbol] [symbol] [symbol] [symbol] , MOCKBa, 2010. 48 c/M. D. Ardatskaya. Clinical use of dietary fibers, Moscow, 2010, 48 pages).

They include substances of polysaccharide nature, such as lactulose, inulin, alginates, fructooligosaccharides, chitin, pectins, acacia, gums, lignin.

It was indeed established that the use of polysaccharide prebiotics notably facilitates recovery after intestinal infections accompanied with dysbiosis. Some prebiotics, for example lignin, chitosan, are also capable of sorbing toxic substances which create as a result of inadequate digestion, and thus prebiotics are more useful in the treatment of intestinal dysbacteriosis.

Furthermore, they perform yet another very important function of normalizing fat and lipid exchange. Particularly, it was established that a prebiotic based on guar gum, known in Ukraine under the trademark “Guarem”, is used for the correction of the state in patients with diabetes, metabolic syndrome and cardiovascular diseases accompanied with hyperlipidemia.

Yet some prebiotics, for example, lactulose, are capable of decreasing toxic effect of ammonia created in excess in case of liver disorders, and have neurotoxic effect. (op cit.: Bengmark S. Colonic food: pre- and probiotics. Am J Gastroenterol 2000; 95 (1) Suppl: S5-7).

However, prebiotics can not totally resolve the problem with disruption of normal microflora and the intoxication syndrome which often accompanies these states either.

Therefore constant attempts have been made to involve other mechanisms of influence on normalization of intestinal microflora and desintoxication. Particularly, it is known that the use of certain silica-based substances may improve the state of microflora in patients with dysbiosis (op cit.: [symbol] . A. [symbol] , H. B. [symbol] , E. Φ. [symbol] , H [symbol] . [symbol] . [symbol] [symbol] oπeKa: [symbol] [symbol] [symbol] [symbol] [symbol] [symbol] [symbol] TpaKTa. [symbol] . [symbol] [symbol] .// [symbol] .-2002.-No 16. C.32/I. A. Zupanets, N. V. Bezdetko, E. F. Grintsov, N. P. Bezuglaya. Pharmaceutical care: Symptomatic treatment of gastrointestinal functional disorders. Meteorism. Dysbacteriosis//Provisor. 2002. Issue no. 16, page 32; [symbol] H. B., [symbol] B. B. COBpeMeHHble [symbol] K [symbol] [symbol] [symbol] ( [symbol] [symbol] ).-K. 2000.-28 c/Kharchenko N. V., Chernenko V. V. Modern approaches to the correction of intestinal dysbiosis (guide to practitioners). Kiev, 2000, 28 pages). To this end, use is made of polymethylsiloxanes, such as simethicone and dimethicone, especially when dysbiosis is accompanied with the symptoms affecting the quality of life, such as, for example, colics, meteorism, defecation difficulties, etc. Toxins bound by a sorbent generally lose their activity and are naturally removed along with the sorbent. Thus, the intoxication syndrome is overcome, and the decrease in the antigen load on immunocompetent cells is achieved, which contributes to compensation of immunodeficiency (secondary) resulting from the development of a pathologic process. Actually, the normalizing influence of hydrogel of methyl-silicic acid on the microflora in a number of clinical situations (op cit.: A. M. [symbol] , O. [symbol] . [symbol] , A. A. [symbol] , O. H. [symbol] . [symbol] [symbol] [symbol] [symbol] B [symbol] [symbol] [symbol] [symbol] y [symbol] C [symbol] [symbol] // [symbol] [symbol] [symbol] >>.-2006-No 1(2).—C. 50-53./A. M. Boyarskaya, O. I. Osadchaya, A. A. Zhernov, O. N. Kovalenko. The use of enterosorbent Enterosgel in the complex treatment of intestinal dysbiosis in children suffering from burn disease//Urgent treatment medicine. 2006. Issue no. 1(2), pages 50-53) is particularly due to the suppression of the activity of pathogenic microorganisms, which thus, figuratively speaking, gives a chance for faster growth of beneficial bacteria.

However, as researches have shown, their efficiency is insignificant (op cit.: Metcalf T J, Irons T G, Sher L D, Young P C. Simethicone in the treatment of infant colic: A randomized, placebo-controlled, multicenter trial//Pediatrics, 1994; 94:29-34.). It is known that other silicon derivatives possess the ability to improve the state of microflora and the general state of a patient with dysbacteriosis. As an example may serve the use of an enterosorbent under the trade name “Enterosgel” which is based on hydrogel of methyl-silicic acid (hydrogel of polymethylsiloxane) (op cit.: [symbol] [symbol] B. H., [symbol] [symbol] . [symbol] [symbol] [symbol] [symbol] [symbol] [symbol] [symbol] [symbol] // [symbol] [symbol] [symbol] [symbol] [symbol] [symbol] [symbol] [symbol] [symbol] [symbol] [symbol] [symbol] . —K., 2007.—c. 76-79/Chernobrovyi V. N., Paliy I. G. The use of Enterosgel for the treatment of intestinal dysbacteriosis//Medical and biological aspects of use of an enterosorbent Enterosgel for the treatment of various diseases. Kiev, 2007, pages 76-79). However, even these preparations are not capable of resolving the problem with treatment of such patients.

Furthermore, it is known that hydrogel of methyl-silicic acid can also bind microorganisms. Therefore a question arises as to whether beneficial probiotics would not also be bound on the surface of a sorbent, which remains open, and therefore its use in combination with eubiotics in the form of hydrogel is hardly possible as opposed to xerogel, namely a dehydrated polymer of methyl-silicic acid. However, there is still no information on the sorbtion characteristics of xerogel upon administration of probiotics, while specialists in the field are well aware of the fact that the introduction of additives to compositions of different enterosorbents results in new properties of the end products. However, such an introduction may be associated with serious disadvantages, such as considerable worsening of sorption acitivity in sorbents, which significantly limits the field of their application.

The methods of combined use of pre- and probiotics, as well as prebiotics with other preparations have been known in the art.

Particularly, UA patent no. 82774 (published on May 12, 2008, Bulletin no. 9) discloses a process for the preparation and use of eubiotics (probiotics) with an enterosorbent based on hydrogel of methyl-silicic acid (i.e. polymethylsiloxane hydrate). At the same time, patent no. 82774 completely lacks data on how the sorbent interacts with eubiotics which are microorganisms. Such a solution is not obvious from the technical standpoint since hydrogel of methyl-silicic acid comprises water molecules in the presence of which probiotics activate and start propagating, which certainly creates technological difficulties in the production, standardization based on microbial number, storage and use of the product. Therefore it is important that preparations for normalization of the intestinal microflora are capable of solving the two key taks:
normalizing the content and characteristics of the intestinal microflora, and
decreasing manifestations of intoxication which results from a number of factors.

### SUMMARY OF THE INVENTION

The problem solved by the invention is to create a composite enterosorbent by altering its content, which would be capable of facilitating a complex solution of problems related to damaged contents and balance of the intestinal microflora resulting from various factors, and to decrease manifestations of the intoxication syndrome.

The said problem is solved by a composite enterosorbent according to the invention, which is based on a silicon polymer selected from the group comprising polymethylsiloxane xerogel or hydrogel of methyl-silicic acid (polymethylsiloxane polyhydrate) and additionally comprises at least one polysaccharide which may be selected from the group including lactulose, inulin, lignin, fructooligosaccharides, alginic acid and its salts, chitosan, pectin, acacia, beta-glucan.

Thus, a composition comprising at least one of the mentioned enterosorbents and a polysaccharide is capable of complex action lying in the restoration of the normal state of the intestinal microflora and the decrease in manifestations of the intoxication syndrome.

The first difference in this composition lies in that the preparation comprises 0.1 to 10 parts by weight of polysaccharide per 1 part by weight of polymethylsiloxane recomputed for xerogel. This composition is suitable for the treatment of those patients with dysbacteriosis which manifest marked intoxication syndrome, particularly in the background of acute intestinal infections.

The second difference lies in that the preparation comprises from 10% to 90% of water. This composition is the most suitable for the long-term treatment of patients, especially of children and advanced age patients, as well as patients undergoing a course of treatment with antibiotics.

The third additional difference lies in that the preparation may be used in the form of a powder for the preparation of capsules or tablets, or in the form of a water solution (suspension).

It is obvious that polymethylsiloxane may be included in the proposed preparation in the form of pharmaceutically acceptable carriers, and that a polysaccharide can also be used in the form of alkaline and alkaline-earth metal salts or water-soluble derivatives which usually comprise an ethyl, methyl, phenyl or acetyl radical of polysaccharides.

### DETAILED DESCRIPTION OF THE INVENTION

The invention is further disclosed in the description of methods for obtaining the preparation and, particularly, experimental dosage forms; description of experiments performed on laboratory models and the obtained results in comparison with the results obtained for traditional preparations, and in the use guide for the treatment with the proposed preparation.

### (1) Methods for Obtaining the Preparation (Particularly of Experimental Dosage Forms)

In all cases, as raw material use was made of both available pharmacopoeia preparations, and of the mentioned substances as chemical reagents having the quality no less than “cp” (chemically pure).

Hydrogel of polymethylsiloxane is obtained with the use of generally known techniques with the subsequent addition of water solution of polysaccharide or a solution of polysaccharide with a respective concentration obtained in the organic solution medium. Following dispersion and the subsequent homogenization of the mixture, a product in the paste- or powder-like form (after drying the product until reaching its constant mass) is obtained.

A specialist in the field would appreciate that calculation of the respective concentrations is made according to the known methods.

Experimental dosage forms in the form of a paste were prepared by mixing hydrogel of methyl-silicic acid and a water solution of polysaccharide. The most efficient ratio of components in order to obtain a paste is 70% and, respectively, 30%. These paste-like preparations were then administered to dysbacteriosis-induced animals in the amounts indicated below.

### Example 1

Preparation of a paste-like product with different ratio of hydrogel of methyl-silicic acid and a water solution of polisaccharide. 30 g of lactulose solution in water is added to 70 g of hydrogel of methyl-silicic acid.

### Example 2

Preparation of a paste-like product with different ratio of hydrogel of methyl-silicic acid and a water solution of polisaccharide. 30 g of sodium alginate solution in water of is added to 70 g of hydrogel of methyl-silicic acid.

### Example 3

Preparation of a paste-like product with different ratio of hydrogel of methyl-silicic acid and a water solution of polisaccharide. 30 g. of 5% water solution of inulin is added to 70 g. of hydrogel of methyl-silicic acid.

Specialists in the field will appreciate that the manufacture of traditional solid dosage forms (tablets, capsules or suppositories) with a specific content of therapeutic agents and, if necessary, well-known additives is based on standard technologies.

### (2) Examples of Practical Embodiments of the Invention

The efficiency of the preparation was experimentally studied according to the officially recommended method (see [symbol] [symbol] . [symbol] [symbol] [symbol] . [symbol] : 2010. 62 c./Methodical guidelines. Pre-clinical study of enterosorbents. Kiev, 2010, 62 pages) on the experimental diarrhea model in rats. Rats were intragastrically administered the Salmonella tiphimurium culture in the amount of 109 cells/ml at the rate of 2 ml per 100 g of the animal body weight. The composite enterosorbent obtained according to example 1 was administered in 24 hours following the infection in the dosage of 100 g of the paste per animal. In 24 hours a coprogram of the animals was analysed. A series of other experiments was run in a similar manner. Their results are provided in table 1 below.

Also, according to these recommendations, adsorption properties of the composite entersorbent were studied by spectrophotometry determining changes in the concentration of the adsorbate (high molecular substances) after storing the mixture (adsorption complex) until reaching the state of adsorption equilibrium. The obtained results are provided in table 2.

The obtained data were statistically processed. The mark (*) following digits in the table means that the statistical discrepancy p is less than 0.05 as compared to the control (upon use of polymethylsiloxane in the form of a paste).

TABLE 1
Comparative data on therapeutic efficiency of the preparations

| | Before | | | |
|---|---|---|---|---|
| Microflora | treatment | After treatment | | P1-2 |
| | | Polymethyl- | Polymethyl- | |
| | | siloxane | siloxane | |
| | | hydrate | hydrate | |
| | | (Enterosgel) (1) | 70%/30% | |
| | | | 1% water solution | |
| | | | of lactulose (2) | |
| General | 102-106 | 108-109 | 109-1010 | * |
| number of | | | | |
| E.coli | | | | |
| Hemolyzing | 20-60% | 0 | 0 | * |
| E.coli | | | | |
| Salmonella | 1010-1012 | 105-106 | 103-104 | * |
| Lactobacteria | 105-107 | 107-108 | 108-109 | * |
| Bifidobacteria | 104-105 | 105-107 | 107-108 | * |

Evaluation of the data provided in table 1 shows that polymethylsiloxane hydrate is capable of decreasing manifestations of dysbiotic changes in the intestine caused by experimental salmonellosis. However, its activity is notably higher in combination with a prebiotic, which is indicative of both apparent propagation of lacto- and bifidobacterial colonies, and of the decrease in the colonies of salmonellas.

Despite of the prebiotic effect, it is important that adsorption properties of a composite enterosorbent do not weaken. The performed studies have shown that such properties did not only decrease, but even increased depending on the components ratio in the composition, which makes a composite enterosorbent even more attractive for clinical use.

TABLE 2
Adsorption properties of the preparations

| | Sorption | Sorption capacity |
|---|---|---|
| | capacity toward | toward methyl |
| | Congo red, | orange, mg/g |
| | mg/g (a < 0.01) | (a < 0.01) |
| Polymethylsiloxane (methyl-silicic | 3.2 | 2.6 |
| acid) hydrate 70%/30% water (control) | | |
| Polymethylsiloxane (methyl-silicic | 3.6 | 3.7 |
| acid) hydrate 70%/30% 1% water | | |
| solution of lactulose | | |
| Polymethylsiloxane (methyl-silicic | 3.2 | 3.7 |
| acid) hydrate 70%/30% 10% water | | |
| solution of lactulose | | |
| Polymethylsiloxane (methyl-silicic | 3.4 | 3.1 |
| acid) hydrate 70%/30% 1% water | | |
| solution of inulin | | |
| Polymethylsiloxane (methyl-silicic | 3.0 | 3.6 |
| acid) hydrate 70%/30% 10% water | | |
| solution of inulin | | |
| Polymethylsiloxane (methyl-silicic | 3.6 | 4.6 |
| acid) hydrate 70%/30% 1% water | | |
| solution of sodium alginate | | |
| Polymethylsiloxane (methyl-silicic | 3.8 | 4.8 |
| acid) hydrate 70%/30% 10% water | | |
| solution of sodium alginate | | |
| Polymethylsiloxane (methyl-silicic | 3.8 | 4.4 |
| acid) hydrate 70%/30% 1% water | | |
| solution of chitosan | | |
| Polymethylsiloxane (methyl-silicic | 4.8 | 4.4 |
| acid) hydrate 70%/30% 10% water | | |
| solution of chitosan | | |
| Polymethylsiloxane (methyl-silicic | 3.5 | 3.2 |
| acid) hydrate 70%/30% 1% water | | |
| solution of acacia | | |
| Polymethylsiloxane (methyl-silicic | 4.0 | 3.4 |
| acid) hydrate 70%/30% 10% water | | |
| solution of acacia | | |
| Polymethylsiloxane (methyl-silicic | 3.2 | 2.6 |
| acid) hydrate 70%/30% 0.9% water | | |
| solution of lactulose | | |
| Polymethylsiloxane (methyl-silicic | 3.0 | 2.4 |
| acid) hydrate 70%/30% 10.1% water | | |
| solution of lactulose | | |
| Polymethylsiloxane (methyl-silicic | 3.2 | 2.4 |
| acid) hydrate 70%/30% 0.9% water | | |
| solution of inulin | | |
| Polymethylsiloxane (methyl-silicic | 3.2 | 2.6 |
| acid) hydrate 70%/30% 10.1% water | | |
| solution of inulin | | |
| Polymethylsiloxane (methyl-silicic | 3.2 | 2.7 |
| acid) hydrate 70%/30% 0.9% water | | |
| solution of sodium alginate | | |
| Polymethylsiloxane (methyl-silicic | 3.2 | 2.6 |
| acid) hydrate 70%/30% 10.1% water | | |
| solution of sodium alginate | | |
| Polymethylsiloxane (methyl-silicic | 3.2 | 2.5 |
| acid) hydrate 70%/30% 0.9% water | | |
| solution of chitosan | | |
| Polymethylsiloxane (methyl-silicic | 3.2 | 2.6 |
| acid) hydrate 70%/30% 10.1% water | | |
| solution of chitosan | | |
| Polymethylsiloxane (methyl-silicic | 3.2 | 2.6 |
| acid) hydrate 70%/30% 0.9% water | | |
| solution of acacia | | |
| Polymethylsiloxane (methyl-silicic | 3.2 | 2.6 |
| acid) hydrate 70%/30% 10.1% water | | |
| solution of acacia | | |

Evaluation of the data provided in table 1 shows that synergistic effect of a composite enterosorbent is present when the components ratio is 1 part by weight of xerogel of polydimethylsiloxane to 0.1-10 parts by weight of polysaccharide.

Recommendations on use of the proposed preparation are based on experimentally established eubiotic properties. Therefore, it should be used for the prevention of the development of dysbacteriosis and for the treatment of various complications, especially those of intoxication genesis.

It is evident that therapists may indicate the proposed preparation in combination with certain other above-mentioned agents for the treatment of gastrointestinal tract, for example protone pump inhibitors, H2-histamine blockers, intestinal bactericidal agents, other sorbents, for example those based on activated carbon.

### INDUSTRIAL APPLICABILITY

The ingredients of the enterosorbent are commercially available on the pharmaceutical market and have been granted respective marketing authorisations. Therefore, following the official trials, the preparation may be used for the treatment of patients with gastrointestinal diseases, including diseases of infectious and non-infectious genesis, in any dosage form, i.e.

in the form of tablets or capsuls—for administration per os,

in the form of suppositories—for rectal administration,

in the form of a gel, paste or suspensions—for administration per os

1. A composite enterosorbent based on a silicon polymer selected from the group comprising xerogel of methyl-silicic acid or hydrogel of methyl-silicic acid, wherein the composite enterosorbent comprises at least one polysaccharide selected from the group including lactulose, inulin, lignin, fructooligosaccharides, alginic acid in the form of its pharmaceutically acceptable salts, chitosan, pectin, acacia, beta-glucan.

2. The composite enterosorbent according to claim 1, comprising 0.1 to 10 parts by weight of polysaccharides per 1 part by weight of hydrogel or xerogel of methyl-silicic acid.

3. The composite enterosorbent according to claim 2, comprising from 10% to 90% of water.

4. The composite enterosorbent according to claim 1, which is in the form of a powder for the preparation of capsules or tablets.

5. The composite enterosorbent according to claim 1, which is in the form of a paste.

6. The composite enterosorbent according to claim 1, which is in the form of a water solution.

7. A method of preparation of capsules or tablets, comprising providing the composite enterosorbent according to claim 1 in the form of a powder, and forming capsules or tablets using the composite enterosorbent.
